(19) United States
(12) Patent Application Publication
(10) Pub. No.: US 2008/0090248 A1
(43) Pub. Date: Apr. 17, 2008

(54) Surface-plasmon resonance-based methods for identifying kinases and their substrates

(76) Inventor: Magdalena Bynum, San Jose, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/546,645

(22) Filed: Oct. 11, 2006

(51) Int. Cl. G01N 33/53; C12Q 1/48

(52) U.S. Cl. 435/7.1; 435/15

(57) ABSTRACT

An assay method is provided. In certain embodiments, the method includes: a) contacting a sample with a polypeptide that is linked to a surface of a surface plasmon resonance sensor under kinasing conditions to produce a sample-contacted substrate; b) separating the sample and the sample-contacted substrate to produce a sample-separated substrate; and c) assessing phosphorylation of the polypeptide using surface plasmon resonance. Kits and systems for performing the instant methods are also provided.

### BACKGROUND

Protein phosphorylation is a major regulatory mechanism for a wide range of diverse cellular processes, including cell division, protein synthesis, signal transduction and transcriptional regulation. It is thought that approximately one third of the proteins in a mammalian cell are phosphorylated and about 510 protein kinases are encoded by the human genome (Kostich et al, Genome Biol. 2002 3: 43.10-43.12). Phosphorylation is reversible, enabling cells to respond to signals in their environment. Further, protein kinase activity plays a role in the development and maintenance of many diseases such as cancer, diabetes and inflammation (Blume-Jensen et al Nature 2001 411:355-365). In a cell, protein kinases use ATP or GTP as a phosphate donor to phosphorylate serine, threonine or tyrosine residues on a target protein.

### SUMMARY OF THE INVENTION

An assay method is provided. In general terms, the method includes: a) contacting a sample with a polypeptide that is linked to a surface of a surface plasmon resonance sensor under kinasing conditions to produce a sample-contacted substrate; b) separating the sample and the sample-contacted substrate to produce a sample-separated substrate; and c) assessing phosphorylation of the polypeptide using surface plasmon resonance. Kits and systems for performing the instant methods are also provided.

Embodiments of the invention may be employed in a variety of assays for identifying protein kinases or their substrates. For example, in one embodiment, the method may be employed to identify a protein kinase for a known substrate. In another embodiment, the method may be employed to identify a substrate for a known kinase.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 schematically illustrates a first embodiment of the instant method.

FIG. 2 schematically illustrates a second embodiment of the instant method.

FIG. 3 illustrate exemplary results obtained using one embodiment of the instant methods.

### DEFINITIONS

The term “sample” as used herein relates to a material or mixture of materials, typically, although not necessarily, in fluid form, e.g., aqueous or in solvent, containing one or more components of interest. Samples may be derived from a variety of sources such as from food stuffs, environmental materials, a biological sample such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components). In certain embodiments, a sample may contain an isolated polypeptide, e.g., a single kinase, or a plurality of isolated polypeptides, e.g., a plurality of kinases.

A “biopolymer” is a polymer of one or more types of repeating units, regardless of the source (e.g., biological (e.g., naturally-occurring, obtained from a cell-based recombinant expression system, and the like) or synthetic). Biopolymers may be found in biological systems and particularly include polypeptides and polynucleotides, including compounds containing amino acids, nucleotides, or a mixture thereof.

The terms “polypeptide” and “protein” are used interchangeably throughout the application and mean at least two covalently attached amino acids, which includes proteins, polypeptides, oligopeptides and peptides. A polypeptide may be made up of naturally occurring amino acids and peptide bonds, synthetic peptidomimetic structures, or a mixture thereof. Thus “amino acid”, or “peptide residue”, as used herein encompasses both naturally occurring and synthetic amino acids. For example, homo-phenylalanine, citrulline and norleucine are considered amino acids for the purposes of the invention. “Amino acid” also includes imino acid residues such as proline and hydroxyproline. The side chains may be in either the D- or the L-configuration.

In general, biopolymers, e.g., polypeptides or polynucleotides, may be of any length, e.g., greater than 2 monomers, greater than 4 monomers, greater than about 10 monomers, greater than about 20 monomers, greater than about 50 monomers, greater than about 100 monomers, greater than about 300 monomers, usually up to about 500, 1000 or 10,000 or more monomers in length. “Peptides” and “oligonucleotides” are generally greater than 2 monomers, greater than 4 monomers, greater than about 10 monomers, greater than about 20 monomers, usually up to about 10, 20, 30, 40, 50 or 100 monomers in length. In certain embodiments, peptides and oligonucleotides are between 5 and 30 amino acids in length.

The terms “polypeptide” and “protein” are used interchangeably herein. The term “polypeptide” includes polypeptides in which the conventional backbone has been replaced with non-naturally occurring or synthetic backbones, and peptides in which one or more of the conventional amino acids have been replaced with one or more non-naturally occurring or synthetic amino acids. The term “fusion protein” or grammatical equivalents thereof references a protein composed of a plurality of polypeptide components, that while typically not attached in their native state, typically are joined by their respective amino and carboxyl termini through a peptide linkage to form a single continuous polypeptide. Fusion proteins may be a combination of two, three or even four or more different proteins. The term polypeptide includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, and the like.

The term “capture agent” refers to an agent that binds a target molecule through an interaction that is sufficient to permit the agent to bind and concentrate the target molecule from a homogeneous mixture of different molecules. The binding interaction is typically mediated by an affinity region of the capture agent. Typical capture agents include any moiety that can specifically bind to a target molecule. In certain embodiments, a polypeptide, e.g., an antibody protein, may be employed. Capture agents usually “specifically bind” a target molecule. Accordingly, the term “capture agent” refers to a molecule or a multi-molecular complex which can specifically bind a target molecule, e.g., a phosphorylated polypeptide, with a dissociation constant (K_{D}) of less than about 10⁻⁶ M (e.g., less than about 10⁻⁷ M, less than about 10⁻⁸ M, less than about 10⁻⁹ M, less than about 10⁻¹⁰ M, less than about 10⁻¹¹ M, less than about 10⁻¹² M, to about usually up to about 10⁻¹⁶ M) without significantly binding to other target molecules, e.g., an unphosphorylated version of the same polypeptide.

The term “specific binding” refers to the ability of a capture agent to preferentially bind to a particular target molecule that is present in a homogeneous mixture of different target molecule. In certain embodiments, a specific binding interaction will discriminate between desirable and undesirable target molecules in a sample, typically more than about 10 to 100-fold or more (e.g., more than about 1000- or 10,000-fold).

The term “capture agent/target complex” is a complex that results from the specific binding of a capture agent with a target, i.e., a “binding partner pair”. A capture agent and an target for the capture agent will usually specifically bind to each other under “conditions suitable for specific binding”, where such conditions are those conditions (in terms of salt concentration, pH, detergent, protein concentration, temperature, etc.) which allow for binding to occur between capture agents and targets to bind in solution. Such conditions, particularly with respect to proteins and antibodies, include those described in Harlow and Lane (Antibodies: A Laboratory Manual Cold Spring Harbor Laboratory, Cold Spring Harbor, N.Y. (1989)) and Ausubel, et al (Short Protocols in Molecular Biology, 5th ed., Wiley & Sons, 2002).

As used herein, “binding partners” and equivalents thereof refer to pairs of molecules that can be found in a capture agent/target complex, i.e., exhibit specific binding with each other.

The phrase “surface-bound polypeptide” refers to a polypeptide that is immobilized on a surface of a substrate. In certain embodiments, the polypeptides employed herein may be present on a surface of the same support, e.g., a sensor.

The term “pre-determined” refers to an element whose identity is known prior to its use. For example, a “pre-determined kinase” is a kinase whose identity is known prior to use. An element may be known by name, sequence, molecular weight, its function, or any other attribute or identifier. In some embodiments, the term “polypeptide of interest”, i.e., a known polypeptide that is of interest, is used synonymously with the term “pre-determined polypeptide”.

The term “antibody protein” is used herein to refer to a capture agent that has at least an epitope binding domain of an antibody. These terms are well understood by those in the field, and refer to a protein containing one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions. Types of antibodies, including antibody isotypes, monoclonal antibodies and antigen-binding fragments thereof (e.g., Fab, Fv, scFv, and Fd fragments, chimeric antibodies, humanized antibodies, single-chain antibodies, etc) are known and need not be described in any further detail.

An “array,” includes any one-dimensional, two-dimensional or substantially two-dimensional (as well as a three-dimensional) arrangement of addressable regions bearing a particular chemical moiety or moieties (e.g., biopolymers such as polynucleotide or oligonucleotide sequences (nucleic acids), polypeptides (e.g., proteins), carbohydrates, lipids, etc.) associated with that region. In the broadest sense, the arrays are arrays of polymeric binding agents, where the polymeric binding agents may be any of: polypeptides, proteins, nucleic acids, polysaccharides, synthetic mimetics of such biopolymeric binding agents, etc. In certain embodiments, the arrays are arrays of polypeptides that are kinase targets.

Any given substrate may carry one, two, four or more or more arrays disposed on a front surface of the substrate. Depending upon the intended use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, photolithographic array fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm. In embodiments that employ surface plasmon resonance detection, the detected light may have a wavelength in the range of 500 nm to 2000 nm, e.g., 600 nm to 1600 nm or 700 nm to 1250 nm. In particular embodiments, a narrow wavelength or single wavelength of light may be detected.

Arrays can be fabricated using drop deposition from pulse jets of either precursor units (such as amino acid or nucleotide monomers) in the case of in situ fabrication, or the previously obtained polymer. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Interfeature areas need not be present particularly when the arrays are made by photolithographic methods.

An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probe” may be the one which is to be evaluated by the other (thus, either one could be an unknown mixture of polynucleotides to be evaluated by binding with the other). A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there exist intervening areas which lack features of interest. An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

The term “mixture”, as used herein, refers to a combination of elements, e.g., kinases, that are interspersed and not in any particular order. A mixture is homogeneous and not spatially separated into its different constituents. Examples of mixtures of elements include a number of different elements that are dissolved in the same aqueous solution, or a number of different elements attached to a solid support at random or in no particular order in which the different elements are not specially distinct. In other words, a mixture is not addressable. To be specific, an array of polypeptides, as is commonly known in the art, is not a mixture of polypeptides because the species of polypeptide on an array are spatially distinct and addressable.

“Isolated” or “purified” generally refers to isolation of a substance (compound, polynucleotide, protein, polypeptide, polypeptide composition) such that the substance comprises a significant percent (e.g., greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90-95% of the sample. Techniques for purifying polynucleotides and polypeptides of interest are well-known in the art and include, for example, ion-exchange chromatography, affinity chromatography and sedimentation according to density. Generally, a substance is purified when it exists in a sample in an amount, relative to other components of the sample, that is not found naturally.

The term “assessing” includes any form of measurement, and includes determining if an element is present or not. The terms “determining”, “measuring”, “evaluating”, “assessing” and “assaying” are used interchangeably and may include quantitative and/or qualitative determinations. Assessing may be relative or absolute. “Assessing the presence of” includes determining the amount of something present, and/or determining whether it is present or absent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

If one composition is “bound” to another composition, the compositions do not have to be in direct contact with each other. In other words, bonding may be direct or indirect, and, as such, if two compositions (e.g., a substrate and a polypeptide) are bound to each other, there may be at least one other composition (e.g., another layer) between to those compositions. Binding between any two compositions described herein may be covalent or non-covalent. The terms “bound” and “linked” are used interchangeably herein.

The term “kinasing conditions” refers to reaction conditions that provide for phosphorylation of a kinase substrate if a kinase and a kinase substrate are present. Suitable kinasing conditions include a source of phosphate, e.g., ATP or GTP, buffer and salt, e.g., MgCl₂ or NaCl and any required cofactors.

The term “phosphorylation” refers to the covalent addition of a phosphate group onto an amino acid of a polypeptide. For example, a phosphate group may be added to the hydroxy group of serine, threonine or tyrosine residue, to produce phosphoserine, phosphothreonine or phosphotyrosine, respectively.

The term “surface plasmon resonance” refers to a detectable electromagnetic phenomenon in which an alteration in a polypeptide can be detected by observing a change in total internal reflectance of a prism coated with a thin metal film.

Other definitions of terms appear throughout the specification.

### DETAILED DESCRIPTION

An assay method is provided. In general terms, the method includes: a) contacting a sample with a polypeptide that is linked to a surface of a surface plasmon resonance sensor under kinasing conditions to produce a sample-contacted substrate; b) separating the sample and the sample-contacted substrate to produce a sample-separated substrate; and c) assessing phosphorylation of the polypeptide using surface plasmon resonance. Kits and systems for performing the instant methods are also provided.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Methods for Identifying Kinases and Their Substrates

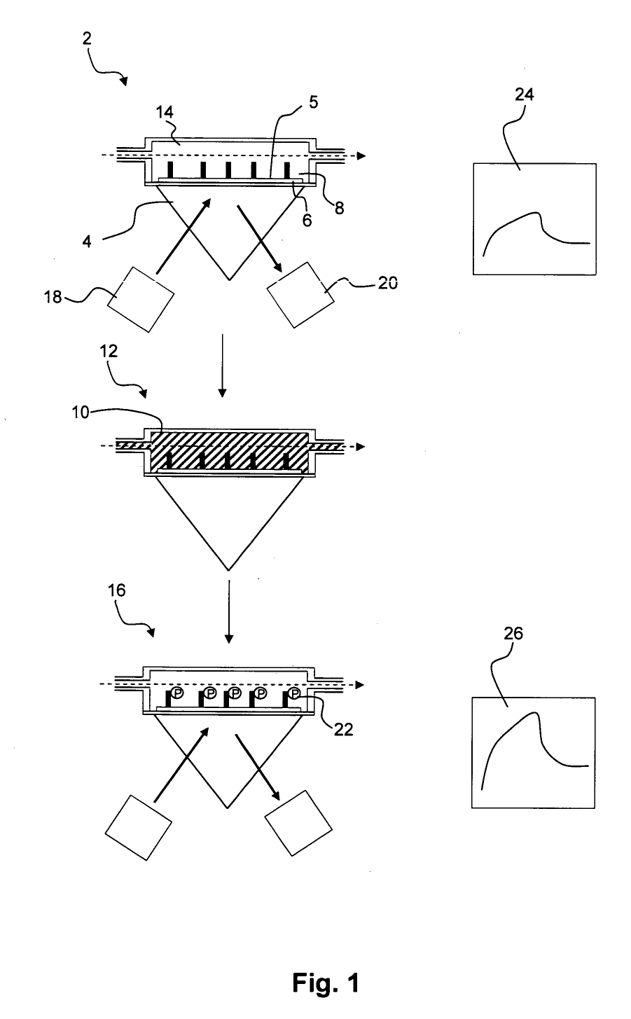

One example of the instant methods is set forth in FIG. 1. With reference to FIG. 1, a surface plasmon resonance sensor 2 comprising a prism 4 having a sensing surface 5 having a surface-linked thin metal layer 6 and a surface-bound polypeptide 8. Polypeptide 8 is contacted with a sample 10 under kinasing conditions to produce a sample-contacted sensor 12. As illustrated in FIG. 1, sensor 2 may in certain embodiments contain a reaction chamber 14, e.g., a flow cell, in which sample can be contacted with the sensing surface of the sensor. After the sample has been contacted with the sensor, the sample is separated from the sensor to produce a sample-separated sensor 16. Phosphorylation of polypeptide 8 is evaluated using surface plasmon resonance, i.e., by detecting reflected light from the substrate surface using a light emitter 18 to illuminate the then metal layer and a detector for detecting light 20 reflected from the metal layer. Any difference in the angle or intensity of the light reflected from the sensing surface of the prism indicates that the surface-bound polypeptide may be phosphorylated by the sample. For example, in the embodiment shown in FIG. 1, the sample contains a kinase for the surface-bound polypeptide, and contact of the sample with the surface-bound polypeptide under kinasing conditions results in the production of a phosphorylated surface-bound polypeptide 22. Phosphorylated surface-bound polypeptide 22 may be detected by observing a change in total internal reflectance before (24) and after (26) polypeptide 8 is contacted with sample 10.

In certain embodiments, the sample 10 may contain a test polypeptide that may or not be a protein kinase. The amino acid sequences of several thousand protein kinases are deposited in NCBI's Genbank database, and those kinases may be produced and purified using known methods. For example, a test polypeptides, e.g., a kinase, may be produced in bacterial, insect or mammalian cells (see, e.g. Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.) using recombinant methods, and isolated. The identity of the polypeptide in the sample may be known or unknown, and the polypeptide in the sample may be known to be a protein kinase prior to its contact with the surface-bound polypeptide. In certain embodiments, the polypeptide in the sample may not be known to be a protein kinase. In particular embodiments, the substrate for the protein kinase in a sample (i.e., the native cellular target for the protein kinase in a cell) may be unknown.

In certain embodiment, the sample may be a complex sample containing a plurality (e.g., at least 1,000 or at least 10,000) different proteins. The sample may be a cell lysate or a fraction of a cell lysate eluted from a separation device (e.g., a chromatography column). In other embodiments, the sample may contain one or more isolated proteins.

In particular embodiments, the sample may contain a plurality of different protein kinases. For example, the sample may contain at least 2, at least 4, at least 10, at least 20, at least 50, up to 100 or 1000 or more protein kinases.

In certain embodiments, the sample may be an unlabeled sample, e.g., not detectably labeled with an optically- or enzymatically-detectable moiety. As such, in one embodiment, a polypeptide may be produced in a recombinant host cell, isolated from other components of the host cell, and used directly in the instant methods.

If the sample contains a kinase, the kinase may be any type of protein kinase, e.g., a serine/threonine kinase or a tyrosine kinase. In particular embodiments, the protein kinase may be a member of the PTK, AGC, CMGC, CaMK and OPK protein kinase families or one of the 55 subfamilies of these five families, as described in Hanks et al, (Science 1988 241: 42-52), Hanks et al, (FASEB J. 1995 9: 576-596) and Hanks et al, (Methods Enzymeology 1991 200: 38-62). In particular embodiments, the protein kinase may be one of the approximately 510 human protein kinases that are encoded by the human genome or an ortholog thereof from a different mammalian species (e.g., mouse or rat), as described by Kostich et al, (Genome Biol. 2002 3: 43.10-43.12).

The surface-linked polypeptide 8 may be a known kinase substrate (e.g., a substrate that is known to be phosphorylated at a serine, threonine or tyrosine residue) or, in certain embodiments may not be known to be a kinase substrate. Accordingly, in certain embodiments, the surface-linked polypeptide may be a kinase target in that it is known or suspected of being a substrate of protein kinase. In certain embodiments, the surface-linked polypeptide does not contain any phosphate groups (e.g., is not a phosphoprotein).

The surface-linked polypeptide may be selected because it is a known to be or suspected of being a substrate for a protein kinase in a sample. Accordingly, in certain embodiments, the surface-linked polypeptide and the protein kinase of the sample may be selected prior to starting the subject methods. In other embodiments, the surface-linked polypeptide may be known to be a kinase substrate, but the identity of the kinase that phosphorylates the surface-linked polypeptide may be unknown. The surface-linked polypeptide may be a substrate for a protein kinase in the sample.

In certain embodiments, the substrate surface may contain a plurality of different surface-bound polypeptides in the form of an array. A subject array may contain at least 4, at least 8, at least 12, at least 24, at least 96, at least 384, at least 1000, or at least 10,000, up to about 20,000 or more different surface-bound polypeptides. The different surface-bound polypeptides may be known kinase substrates, polypeptides that are not known to be kinase substrates, or a combination of polypeptides are known kinase substrates and polypeptides that are not known to be kinase substrates.

The surface-linked polypeptides may be chosen using any convenient method. For example, the surface-linked polypeptides present on an array may be substrates of protein kinases of a particular signal transduction, developmental or biochemical pathway, protein kinases having similar biological functions, protein kinases of similar size or structure, or they may be known markers for a biological condition or disease. The surface-linked polypeptides may also be chosen at random, or on the availability of polypeptides, e.g., if a polypeptide is available for purchase, for example. In some embodiments, a polypeptide may be chosen purely because it is desirable to know whether a known or unknown kinase for that polypeptide is present in a sample. The protein kinase that phosphorylates a polypeptide does not have to be known for the polypeptide to be present on a subject array.

Further, since the capture agents are chosen using any convenient method, there is no requirement that the surface-bound polypeptide is a substrate for a kinase in the sample. In fact, since the subject methods may be used to determine the presence or absence of a protein kinase in a sample, as well as whether a surface-bound polypeptide may be phosphorylated by a protein kinase in the sample, only a fraction or none of the protein kinases in a sample may have substrate polypeptides on a subject array.

In certain embodiments, an array for use in certain embodiments of the subject methods may include at least two different features containing the same polypeptide. Further, as will be discussed in greater detail below, in certain cases one or more features of a subject array may contain control polypeptides that are not expected to be phosphorylated. In this embodiment, the surface plasmon resonance reading is expected to be the same before and after contacting the sample with the substrate.

A surface plasmon resonance reading may be taken prior to contacting the sample with the substrate to provide a baseline reading.

As noted above, the sample is contacted with the polypeptide under kinasing conditions. In certain embodiments, the sample contains all of the components necessary for kinase activity if a kinase is present in the sample. Such conditions include those described in, e.g., Ausubel, et al., (Short Protocols in Molecular Biology, 3rd ed., Wiley & Sons 1995) and Sambrook et al., (Molecular Cloning: A Laboratory Manual, Third Edition, 2001 Cold Spring Harbor, N.Y.). For example, the sample may contain a source of phosphate (e.g., ATP or GTP or an analog thereof at a concentration of 5 μM to 100 mM, e.g., about 100 μM to 1 mM), salt, e.g., MgCl₂ at a concentration of 1 mM to 100 mM, e.g. about 25 mM, and may be buffered using Tris or HEPES to pH 7.0 to 9.0, e.g., pH 7.4 to pH 8.0. In certain cases, a cofactor for a kinase (e.g., CaCl₂ or a lipid) may also be included in the kinase reagents used. Kinasing conditions may further include maintaining the sample and the surface-bound polypeptide at a temperature that is suitable for kinase activity (e.g., a temperature in the range of 15° C. to 42° C., 30° C. to 40° C. or about 37° C.) for a suitable period of time, e.g., a time period in the range of 5 min to 24 hr, 15 min to 12 hr or 30 min to 4 hr. In certain embodiments, the sample may contain a positive control kinase that is known to be active (i.e., able to transfer phosphate from a source of phosphate onto a surface-bound positive control polypeptide) under the kinasing conditions employed. In certain embodiments, after a certain period of time, the kinase reaction may be terminated by addition of EDTA or the like.

Also as noted above, in certain embodiments, the method may include separating the sample and the surface-bound polypeptide after they have been contacted under kinasing conditions. In this embodiment, the sample may be replaced with a wash buffer that transports any components that are not covalently bound to the substrate (e.g., any kinases in the sample, etc.) away from the substrate. In certain embodiments, this step may include washing with a buffer that does not include the test polypeptide or does not include kinase reagents at a suitable pH (e.g., a pH above pH 8.0 or below pH 7.0) for a suitable period of time, e.g., 5 min to 8 hours. Since many kinases have a high off-rate, stringent wash conditions need not be employed in certain embodiments. In other words, in certain embodiments, the separating step of the instant methods may include kinase disassociation conditions in which any kinases in the sample are disassociated from the surface-bound polypeptide. Such conditions may include contacting the substrate with a wash buffer, e.g., PBS, TBS, or an acid or alkali buffer, to separate any kinases in the sample from any of the surface-bound polypeptides to which they may be bound. Such conditions are known (see, e.g., Benitez et al, Mol. Cell Biochem. 1999 191: 29-33; Benitez Biochem J. 1997 324: 987-94; Benitez et al, Mol. Cell Biochem. 2001 227:31-36 and Benitez et al, Anal. Biochem. 2002 302: 161-8).

In certain embodiments, SPR is performed only after the sample-contacted substrate has been subjected to kinase disassociation conditions, i.e., such that any covalent attachment of a phosphate group, rather than a non-covalent binding interaction between a polypeptide in the sample and the surface-bound polypeptide, can be identified. In certain embodiments, an SPR reading may be done under conditions in which only covalent modifications to the surface-bound polypeptide can be detected. In other words, in certain embodiments, the SPR reading may be done under conditions in which non-covalent interactions between a surface-bound polypeptide and a kinase or other polypeptide in the sample are minimized or not detectable.

As will be described in greater detail below and as illustrated in FIG. 1, a surface plasmon resonance reading may be taken after the sample has been separated from the substrate. In certain embodiments, a surface plasmon resonance reading may be done immediately after separating the sample from the substrate.

In other embodiments a surface plasmon resonance reading may be done after the sample-separated substrate has been contacted with a phosphoprotein-specific capture agent, e.g., an antibody protein that detects phosphorylated polypeptides. In particular embodiments, surface plasmon resonance readings may be taken immediately after the sample has been separated from the substrate and also after the sample-separated substrate has been contacted with a phosphoprotein-specific capture agent. In other words, the presence of the phosphorylated surface-bound polypeptide can be detected by detecting the change in surface plasmon resonance due to the sole addition of the phosphate group and/or due to the binding of an antibody to the phosphate group.

For example, in certain embodiments, any one or more of a variety of labeled anti-phosphotyrosine, anti-phosphoserine or anti-phosphothreonine antibody proteins may be used. Such antibody proteins, which may be polypeptide-specific (i.e., may specifically bind to only single phosphorylated polypeptide but not other polypeptides or a non-phosphorylated form of the polypeptide) or may non-specifically bind a wide range of phosphoproteins. Such antibody proteins may be purchased from a variety of different manufacturers, including Research Diagnostics Inc. (Flanders N.J.), Zymed Laboratories, Inc. (San Francisco, Calif.), PerkinElmer (Torrance, Calif.) and Sigma-Aldrich (St. Louis, Mo.). Specific binding conditions for representative capture agents are known in the art and generally involve incubating the capture agent and surface-bound polypeptide in a binding buffer, e.g., phosphate buffered saline (PBS; 137 mM NaCl, 10 mM phosphate, 2.7 mM KCl, pH 7.4) or Tris buffered saline (10 mM Tris 50 mM NaCl, pH. 7.0) for a period of time, usually from 1 to 12 hours at room temperature or 37° C., for example. Other conditions are that may be employed include those described in, e.g., Harlow and Lane (Antibodies: A Laboratory Manual Cold Spring Harbor Laboratory, Cold Spring Harbor, N.Y. (1989) and Ausubel, et al, Short Protocols in Molecular Biology, 5th ed., Wiley & Sons, (2002)). Following contact with the phosphoproteins-specific capture agent, the substrate may be washed (e.g., using PBS or TBS), prior to reading.

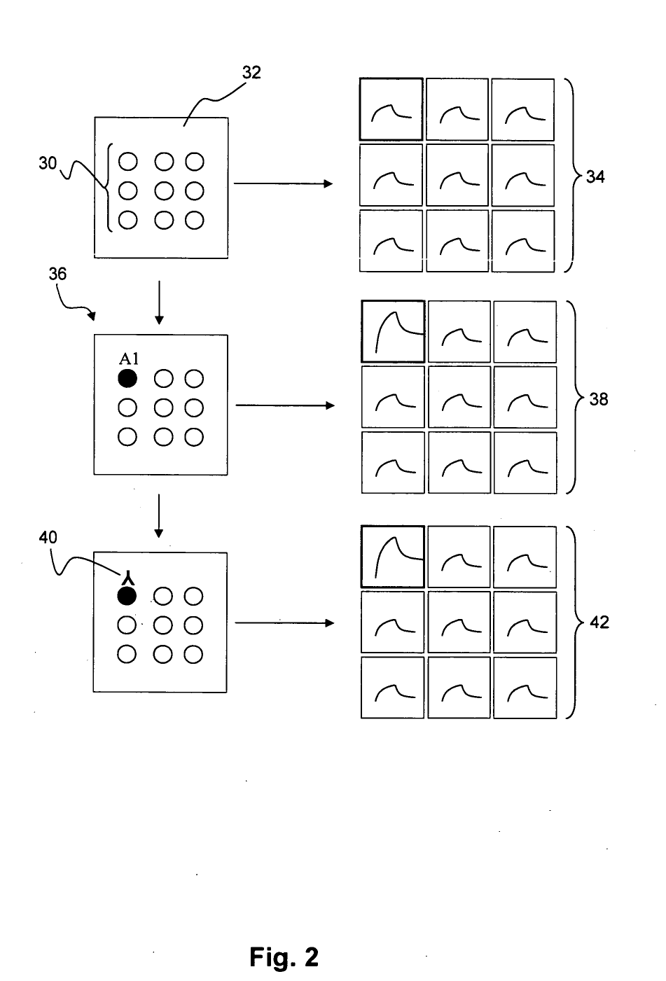

One exemplary embodiment of the instant method is set forth in FIG. 2. In this embodiment, an array of test polypeptides 30 on a substrate 32 is read to produce surface plasmon resonance data 34. This data serves as a baseline to which future data may be compared. The substrate is contacted with a substrate under kinasing conditions to produce a sample-contacted substrate, and the sample is separated from the sample-contacted substrate to produce a sample-separated substrate 36. Optionally, sample-separated substrate 36 may be read to produce surface plasmon resonance data that indicates that the polypeptide at position A1 is phosphorylated by a kinase in the sample. The sample separated substrate may be contacted with a phosphoproteins-specific capture agent 40 and the substrate may be again read to produce surface plasmon resonance data 42 that indicates that the polypeptide at position A1 is phosphorylated by a kinase in the sample.

As noted above, a surface plasmon resonance (SPR) sensor is employed in the subject methods. SPR is produced when a laser beam, linearly polarized parallel to the plane of incidence, impinges onto a prism coated with a thin metal film at an angle that generates an evanescent wave. SPR is most easily observed as a change in the total internally reflected light just past the critical angle of the prism. This angle of minimum reflectivity (denoted as the SPR angle) shifts to higher angles as the molecular weight of the polypeptide that is abound to the metal layer increase (e.g., by the addition of a phosphate group and/or the addition of a phosphate group and a capture agent, e.g., antibody). The shift in the angle can be converted to a measure of the thickness of the adsorbed or added material by using Fresnel calculations and can be used to detect alterations to the polypeptides bound to the capture agents on top of the metal layer. As is well known, SPR may be performed with or without a surface grating (in addition to the prism). Accordingly a subject sensor may contain a grating, and may be employed in other SPR methods other than that those methods explicitly described in detail herein.

In using SPR to test for an alteration in a surface-bound polypeptide, a beam of light from a laser source 18 is directed through a prism 4 onto a subject sensor containing a transparent substrate, which has one external surface covered with a thin film of a metal, which in turn is covered with a surface-bound polypeptide, as discussed above. The SPR angle changes upon molecular modification of the surface-bound polypeptide. By monitoring either the position of the SPR angle or the reflectivity at a fixed angle near the SPR angle, the presence or absence of a phosphorylation event can be detected.

Various types of equipment for using SPR with a biosensor for biological or biochemical or chemical substances are known in the art (and described by Liedberg et al. (1983) Sensors and Actuators 4:299, European Patent Application 0305108 and U.S. Pat. No. 5,374,563, etc.), including grating coupled systems, optical waveguide systems and prism coupled attenuated total reflection systems.

In certain embodiments, a light source (typically a monochromatic light source) is used to illuminate the prism/metal film at an incident angle that is near the SPR angle, and the reflected light is detected at a fixed angle with a CCD camera to produce an SPR image. The SPR image arises from variations in the reflected light intensity from different parts of the sample; these variations are created by any changes in organic film thickness or changes in index of refraction that occur upon adsorption onto the modified gold surface. SPR imaging is sensitive only to molecules in proximity to the surface, therefore unbound molecules remaining in solution do not interfere with in situ measurements.

In certain embodiments, the angles of incidence and reflection are “swept” together through the resonance angle, and the light intensity is monitored as function of angle. Very close to the resonance angle, the reflected light is strongly absorbed by the gold surface, and the reflected light becomes strongly reduced. In other embodiments, the source and detector angles are fixed near the resonance angle at an initial wavelength, and the wavelength is swept to step the resonance point through the fixed angle. The beam is collimated and an entire image of the substrate is captured. In exemplary embodiments, the wavelength of the tunable laser may be between from 0.6 μm to about 0.8 μm (i.e., having a 200 nm sweep), although tunable lasers having other sweeps (e.g., 0.8 μm to 1.0 μm, 1.0 μm to 1.2 μm, 1.2 μm to 1.4 μm, 1.4 μm to 1.6 μm or 1.6 μm to 1.8 μm may also be employed. In one embodiment, a tunable laser having a sweep of 1.45 to 1.65 μm is employed.

An SPR reader is used to accomplish the task of obtaining data from a subject sensor, which readers are generally well known in the art (see U.S. Pat. No. 6,466,323, for example). In one embodiment, polypeptide 8 is bound to the substrate. A liquid is introduced into chamber 14, and, if the surface-bound polypeptide is a substrate for a kinase in the sample, or if a surface-bound polypeptide is recognized by a phosphoprotein-specific capture agent, the mass of the surface-bound polypeptide increases, resulting (for a given incident angle of light in an applied range of beam angles “R”) in a light reflectance angle “θ” where light intensity maximizes, minimizes, or varies.

Evanescent wave producing light having a wavelength of between about 400 nm to about 2.0 μm may used in the subject methods. In exemplary embodiments, the wavelength of light used is from about 0.8 μm to about 1.7 μn, e.g., 0.8 μm to about 1.6 μm. In certain embodiments, the light used is monochromatic light, and the light may be polarized, and in certain embodiments, the wavelength of light used may change, i.e., may “sweep” during reading of a sensor. Accordingly, the light used may not be of a static wavelength. In typical embodiments, the wavelength may sweep between two different wavelengths separated by about 100 nm, about 200 nm, about 300 nm or about 400 nm or more, with the lower wavelength being any of the wavelengths listed above. The light employed in for evanescent wave detection should have a wavelength that is different to that of the light used as a cleaving stimulus, and, as such, should not cleave the capture agents from the substrate. In one embodiment, the evanescent wave-producing light has a wavelength of 1.45 μm to 1.65 μm may be employed, although broadband evanescent wave-producing light of a wide variety of sweeps and wavelengths may be used.

In addition to the above-recited method, a system for performing the method is also provided. The system may contain an SPR sensor having a surface-bound polypeptide, as discussed above, and kinasing reagents. The system may further contain any of the other components described above. For example, in one embodiment, the system may have an SPR sensor that has a flow cell that is fluidically to a reservoir containing kinase reagents, a reservoir containing buffer for separating the sample from the substrate, and/or a reservoir of capture agents, for example.

### Utility

The subject methods may be employed in a variety of diagnostic, drug discovery, and research applications that include, but are not limited to, diagnosis or monitoring of a disease or condition (where an active kinase for a particular polypeptide is a marker for the disease or condition), discovery of drug targets (where a kinase is differentially phosphorylation in a disease or condition and may be targeted for drug therapy), drug screening (where the effects of a drug are monitored by assessing the level of phosphorylation of an polypeptide), determining drug susceptibility (where drug susceptibility is associated with a particular profile of kinase modifications) and basic research (where is it desirable to identify a kinase for a substrate or a substrate for a kinase). In certain embodiments, a collection of different kinases (e.g., at least 10, at least 20, at least 50 or at least 100 different kinases) may be independently tested to determine if they phosphorylate a particular surface-bound polypeptide. In other embodiments, a collection of different surface-bound polypeptides (e.g., at least 10, at least 20, at least 50 at least 100 or at least 1000 or more different surface-bound polypeptides) may be independently tested (e.g., using an array of those polypeptides) to determine if they are phosphorylated by a particular protein kinases.

In particular embodiments, the instant methods may be used to identify kinases in a sample. In these embodiments, a sample is analyzed using the above methods, and the identity of some or all of the kinases in the sample can be determined by the phosphorylation pattern of the surface-bound polypeptides. In certain embodiments, the subject methods may be employed to produce a “profile” of active kinases in a sample.

In certain embodiments, a sample may be analyzed to determine if a particular kinase is present in the sample.

In other embodiments, relative active kinase profile of two or more different samples may be obtained using the above methods, and compared. In these embodiments, the results obtained from the above-described methods are usually normalized to the total amount of protein present in the sample (or a positive control kinase), and compared. This may be done by comparing ratios, as described above, or by any other means. In particular embodiments, the kinase profiles of two or more different samples may be compared to identify active kinases that are associated with a particular disease or condition (e.g., a kinase event that is induced or activated by the disease or condition and therefore may be part of a signal transduction pathway implicated in that disease or condition).

The different samples may consist of an “experimental” sample, i.e., a sample of interest, and a “control” sample to which the experimental sample may be compared. In many embodiments, the different samples are pairs of cell types or fractions thereof, one cell type being a cell type of interest, e.g., an abnormal cell, and the other a control, e.g., normal, cell. If two fractions of cells are compared, the fractions are usually the same fraction from each of the two cells. In certain embodiments, however, two fractions of the same cell may be compared. Exemplary cell type pairs include, for example, cells isolated from a tissue biopsy (e.g., from a tissue having a disease such as colon, breast, prostate, lung, skin cancer, or infected with a pathogen etc.) and normal cells from the same tissue, usually from the same patient; cells grown in tissue culture that are immortal (e.g., cells with a proliferative mutation or an immortalizing transgene), infected with a pathogen, or treated (e.g., with environmental or chemical agents such as peptides, hormones, altered temperature, growth condition, physical stress, cellular transformation, etc.), and a normal cell (e.g., a cell that is otherwise identical to the experimental cell except that it is not immortal, infected, or treated, etc.); a cell isolated from a mammal with a cancer, a disease, a geriatric mammal, or a mammal exposed to a condition, and a cell from a mammal of the same species, preferably from the same family, that is healthy or young; and differentiated cells and non-differentiated cells from the same mammal (e.g., one cell being the progenitor of the other in a mammal, for example). In one embodiment, cells of different types, e.g., neuronal and non-neuronal cells, or cells of different status (e.g., before and after a stimulus on the cells) may be employed. In another embodiment of the invention, the experimental material is cells susceptible to infection by a pathogen such as a virus, e.g., human immunodeficiency virus (HIV), etc., and the control material is cells resistant to infection by the pathogen. In another embodiment of the invention, the sample pair is represented by undifferentiated cells, e.g., stem cells, and differentiated cells.

Accordingly, among other things, the instant methods may be used to link the activity of certain kinases to certain physiological events.

In particular embodiments, the subject methods may be used to establish cellular signaling pathways that are employed to transmit signals in a cell (e.g., from the exterior or interior of the cell to a cell nucleus, or from one protein in a cell to another, directly or indirectly). For example, the subject methods may be employed to determine which kinases in a cell are active at any moment in time, or to identify a substrate for a kinase. The substrates for a particular kinase be identified by virtue of the fact that they should be phosphorylated by the kinase, at the same point in time.

In one embodiment, the invention also provides a method of screening for an agent that modulates kinase activity. The method generally comprises contacting a candidate agent with the sample and assessing the sample according to the above-recited methods. In certain embodiments, the results from this assay may be compared to those of an otherwise identical sample that has not been contacted with the candidate agent. Such a method may be employed to identify an agent that reduces or increases the activity of a particular kinase.

A variety of different candidate agents may be screened by the above methods. Candidate agents encompass numerous chemical classes, though typically they are organic molecules, preferably small organic compounds having a molecular weight of more than 50 and less than about 5000 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc., to produce structural analogs.

Agents that modulate kinase activity typically decrease or increase the amount of a phosphorylation of a substrate (relative to the total amount of that substrate) by at least about 10%, at least about 20%, at least about 50%, at least about 70%, or at least about 90%.

### Kits

Also provided by the subject invention are kits for practicing the subject methods, as described above. The subject kits contain at least a surface plasmon resonance sensor and kinasing reagents, as discussed above. The kit may further contain any of the other components discussed above, e.g., a positive control kinase, a negative control kinase, a phosphopeptide-specific capture agent (e.g., an antibody protein). The various components of the kit may be present in separate containers or certain compatible components may be precombined into a single container, as desired.

In addition to above-mentioned components, the subject kits may further include instructions for using the components of the kit to practice the subject methods, i.e., to instructions for sample analysis. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the subject database, programming and instructions, the kits may also include one or more control analyte mixtures, e.g., one or more control samples for use in testing the kit.

### EXAMPLES

The following examples are put forth so as to provide those of ordinary skill in the art with a complete disclosure and description of how to make and use the present invention, and are not intended to limit the scope of what the inventors regard as their invention. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Kinase assays were run on the Biacore 3000 (Biacore AB, Uppsala, Sweden). Materials used for the Biacore included CM5 sensor chips, N-ethyl-N′-(dimethylaminopropyl)-carbodiimide (EDC), N-hydroxysuccinimide (NHS), ethanolamine hydrochloride (1M), and pH 4.0 acetate, all of which were obtained from Biacore AB. Reagents such as 10 mM HCL, 10 mM NaOH, 0.1% SDS, and 1×PBS (consisting of 137 mM NaCl, 2.7 mM potassium chloride, 10 mM phosphate buffer Na₂HPO₄/KH₂PO₄, pH 7.4), were also used in the experiments. Phosphorylated αₛ₁-casein; dephosphorylated αₛ₁-casein; and purified, monoclonal, anti-phosphoserine antibodies were obtained from Sigma-Aldrich Co., (St. Louis, Mo.). Casein kinase I (CK1) was obtained from Promega, (Madison, Wis.). The kinase assays took place in 25 mM Tris-HCL (pH 7.4), 10 mM MgCl2, and 0.1 mM ATP. Lastly, the protein phosphorylation resulting from the kinase reactions was validated using an Agilent LC/MS Time of Flight mass spectrometer (Palo Alto, Calif.).

Immobilization of αₛ₁-casein on a CM5 chip. αₛ₁-casein is a 23 kDa heterogeneous and hydrophobic protein isolated from bovine milk. Four proteins, αₛ₁, αₛ₂, β, and κ, make up the casein family. αₛ₁-casein has a major and minor component and has 8 phosphorylation sites on its major component. αₛ₁-casein was dissolved to a stock concentration of 2 mg/ml in PBS buffer. The stock solution was then further diluted to 200 μg/ml in buffer of 4.0, 4.5, 5.0, and 5.5 acetate and was tested for preconcentration levels. These tests showed that pH 4.0, which is below the pI of casein (˜4.5), yielded the highest αₛ₁-casein immobilization. After activating the dextran surface of a CM5 chip with EDC and NHS, the phosphorylated αₛ₁-casein was injected for seven minutes at a flow rate of 5 μl per minute. Ethanolamine was then injected to block non-bound reaction sites on the CM5 chip. Phosphorylated αₛ₁-casein will be referred to as (+) αₛ₁-casein from here on.

Dephosphorylated αₛ₁-casein was immobilized under the same conditions. Dephosphorylated αₛ₁-casein will be referred to as (−) αₛ₁-casein from here on.

Casein kinase phosphorylation assay. Casein was phosphorylated using Casein Kinase I, which has dual specificity for threonine and serine residues. This kinase phosphorylates the serine residues of casein. Before immobilizing αₛ₁-casein, the flow cells were rinsed twice with 10 mM HCL, 50 mM NaOH, and 1% SDS to rid their surfaces of unwanted materials (100 μl/min, 6 sec each).

A CM5 chip was loaded as follows: The chip surface was activated with EDC/NHS (20 μl/min, 7 min). The surface of flow cell 1 was blocked with ethanolamine. This was the reference cell that was subtracted from the activity of the other flow cells to account for temperature variation and bulk refractive index changes. A 400 μg/ml solution of (+) αₛ₁-casein was immobilized on the surface of flow cell 2 (7 min, 5 μl/min) and served as the positive control (in reference to the antibody tests). A 200 μg/ml solution of (−) αₛ₁-casein was immobilized on the surfaces of both flow cells 3 and 4.

The buffer for the kinase reaction contained 25 mM Tris-HCL (pH 7.4), 10 mM MgCl₂, 0.1 mM ATP, and 10 u CK1, (where 1 u is the amount of enzyme required to transfer 1 pmol of phosphate per minute at 37° to the casein substrate. Promega Co., Madison, Wis.). After the casein immobilization on the surfaces of three flow cells, the CK1 solution was injected into flow cells 1-3 at 25° (5 μl/min, 30 min). Flow cell 4 was not exposed to the kinase mix and served as a negative control for the antibody tests.

To check phosphorylation, we first injected base through all flow cells to remove adsorbed kinase. Anti-phosphoserine antibodies (50 μg/ml) were then run through all flow cells (3 min, 20 μl/min) with the expectation that flow cell 3 would show binding (whereas before phosphorylation by CK1 it did not).

Determining response from kinase binding and phosphorylation. In order to distinguish the amount of signal that was due to the kinase adsorption versus the amount that was due to casein phosphorylation, an assay was set up to measure the signal level that would result only from the kinase affiliation to αₛ₁-casein. (−) αₛ₁-casein was exposed to CK1 under conditions identical to the CK1 phosphorylation assay except for the presence and absence of ATP (the phosphate group donor).

(−) αₛ₁-casein was diluted to concentrations of 200 μg/ml in pH 4.0 acetate and was immobilized on the surface of two flow cells (5 μl/min, 7 min). Anti-phosphoserine antibodies were run over these flow cells at 50 μg/mL in PBS before the kinase incubation to validate that the antibodies did not bind to (−) αₛ₁-casein (20 μl/min, 3 min). Base injections (100 μl/min, 10 s) flushed the flow cells of bound antibody. The kinase solution that included ATP was then injected thru one flow cells (5 μl/min, 30 min), followed by two pulses of base, and then followed by an injection of anti-phosphoserine antibodies. The same procedure was then followed for the kinase solution that lacked ATP over the other flowcell.

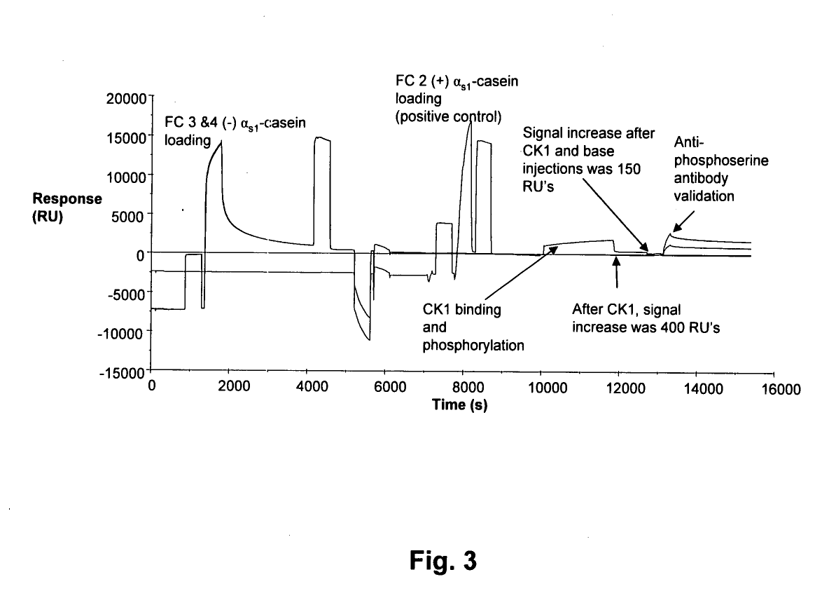

Results of these assays are illustrated in FIG. 3.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An assay method comprising:
a) contacting a sample with a polypeptide that is linked to a surface of a surface plasmon resonance sensor under kinasing conditions to produce a sample-contacted substrate;
b) separating said sample and said sample-contacted substrate to produce a sample-separated substrate; and
c) assessing phosphorylation of said polypeptide using surface plasmon resonance.

2. The assay method of claim 1, wherein said sample is known to contain a kinase.

3. The assay method of claim 1, wherein said sample is known to contain a plurality of kinases.

4. The assay method of claim 1, wherein said sample comprises kinasing reagents.

5. The assay method of claim 1, wherein said substrate surface comprises an array of different polypeptides.

6. The assay method of claim 1, wherein said polypeptide is a known kinase substrate.

7. The assay method of claim 1, wherein said separating includes contacting said sample-contacted substrate with wash buffer that disassociates polypeptides in said sample from said surface-bound polypeptide.

8. The assay method of claim 1, wherein said assessing includes evaluating binding of a phosphoprotein-specific capture agent to said sample-separated substrate.

9. The assay method of claim 8, wherein said phosphoprotein-specific capture agent is an antibody protein.

10. The assay method of claim 9, wherein said antibody protein specifically binds to a phosphorylated polypeptide.

11. The assay method of claim 10, wherein said antibody protein specifically binds to phosphotyrosine, phosphoserine or phosphothreonine residues.

12. A method comprising:
a) analyzing a sample according to the assay method of claim 1 to produce data; and
b) analyzing said data to determine if said polypeptide is phosphorylated.

13. The method of claim 12, wherein said method provides for identification of a substrate for a known kinase.

14. The method of claim 12, wherein said method provides for identification of a kinase for a known substrate.

15. A system comprising:
a) a surface plasmon resonance sensor having a surface-linked polypeptide; and
b) a reservoir of sample comprising kinasing reagents, in fluid communication with said surface plasmon resonance sensor.

16. The system of claim 15, further comprising: c) a reservoir of phosphoprotein-specific capture agent, in fluid communication with said surface plasmon resonance sensor.

17. The system of claim 15, further comprising a c) a reservoir of wash buffer for separating said sample from said surface-linked polypeptide, in fluid communication with said surface plasmon resonance sensor.

18. A kit comprising:
a) kinase reagents; and
b) a surface plasmon resonance sensor comprising a surface-bound polypeptide.

19. The kit of claim 18, further comprising a phosphoprotein-specific capture agent.

20. The kit of claim 18, wherein said capture agent is an antibody.
